(19) United States
(12) Patent Application Publication
(10) Pub. No.: US 2020/0093960 A1
(43) Pub. Date: Mar. 26, 2020

(54) METHODS AND MATERIALS FOR TREATING FISTULAS

(71) Applicant: Mayo Foundation for Medical Education and Research, Rochester, MN (US)

(72) Inventors: Allan B. Dietz, Chatfield, MN (US); William A. Faubion, Rochester, MN (US); Eric J. Dozois, Rochester, MN (US)

(73) Assignee: Mayo Foundation for Medical Education and Research, Rochester, MN (US)

(21) Appl. No.: 16/494,900

(22) PCT Filed: Mar. 21, 2018
(86) PCT No.: PCT/US2018/023616

§ 371 Date: Sep. 17, 2019

Related U.S. Application Data
(60) Provisional application No. 62/474,483, filed on Mar. 21, 2017.

(51) Int. Cl. A61L 27/38; A61K 35/28; A61L 27/18; A61L 27/56; A61K 9/00

(52) U.S. Cl. CPC A61L 27/3834; A61K 35/28; A61K 9/0031; A61L 27/56; A61L 27/18

(57) ABSTRACT

This document provides methods and materials for treating fistulas (e.g., refractory fistulas such as refractory anal fistulas). For example, methods and materials for implanting a synthetic scaffold (e.g., fistula plug) comprising randomly arranged fibers comprising polymers of PGA and TMC and seeded with mesenchymal stem cells (e.g., adipose derived mesenchymal stem cells) located in the spaces between the randomly arranged fibers into a fistula (e.g., refractory anal fistula) of a mammal (e.g., a human) are provided.

### CROSS-REFERENCE TO RELATED APPLICATIONS

This application claims priority to U.S. application Ser. No. 62/474,483, filed on Mar. 21, 2017. The disclosure of the prior application is considered part of the disclosure of this application, and is incorporated in its entirety into this application.

### BACKGROUND

### 1. Technical Field

This document relates generally to medical devices, and particularly to devices, systems, and methods for treating fistulas (e.g., refractory fistulas such as refractory anal fistulas and refractory broncho pleural fistulas).

### 2. Background Information

Unresolved healing is a significant issue in medicine. Failure to heal can lead to ulcers (wounds open to the environment) and abscesses. Abscesses are infected anatomical cavities. A fistula is a type of abscess cavity characterized by a tunnel running between two hollow organs, or between a hollow organ and the surface of the skin. For example, anal fistulas are infected tunnels that develop between the rectum and the skin around the anus. Some anal fistulas are the result of an infection in an anal gland that spreads to the skin. Inflammatory bowel diseases, such as Crohn's disease, also substantially contribute to the formation of fistulas involving the digestive tract. Treatment modalities for anal fistulas depend on the fistula's location and complexity. The general goals of fistula treatments are to achieve complete fistula closure, to prevent recurrence, and to avoid damaging the sphincter muscles which can lead to fecal incontinence. Healing abscessed cavities is a significant challenge.

### SUMMARY

This document provides methods and materials for treating fistulas (e.g., anal fistulas, cryptoglandular fistulas, bronco pleural fistulas, rectal vaginal fistulas, and refractory fistulas such as refractory anal fistulas, refractory cryptoglandular fistulas, refractory bronco pleural fistulas, and refractory rectal vaginal fistulas). For example, this document provides methods and materials for implanting a synthetic scaffold (e.g., fistula plug) comprising randomly arranged fibers comprising polymers of polyglycolic acid (PGA) and trimethylene carbonate (TMC) and seeded with mesenchymal stem cells (e.g., adipose derived mesenchymal stem cells) located in the spaces between the randomly arranged fibers into a fistula (e.g., refractory anal fistula) of a mammal (e.g., a human). One example of such a synthetic scaffold is the GORE® BIO-A® Fistula Plug seeded with mesenchymal stem cells (e.g., adipose derived mesenchymal stem cells) located in the spaces between the randomly arranged fibers.

Despite the development of many different synthetic materials and many different natural biologic materials to treat fistulas, the ability to improve the successful treatment of fistulas, especially refractory fistulas such as refractory anal fistulas, remains an important need for clinicians and patients. As described herein, many different synthetic materials and natural biologic materials were obtained and seeded with adipose derived mesenchymal stem cells. Each of these combinations was assessed in vitro and the lead candidate matrix was studied further in vivo for the ability to successfully treat refractory anal fistulas. Most of the tested materials seeded with adipose derived mesenchymal stem cells as described herein resulted in poor cell seeding as assessed in vitro. One material when seeded as described herein, however, significantly out performed all the other tested materials, resulting in an unexpectedly high level of cell seeding and proliferation as assessed in vitro and the very effective complete healing of 10 out of 12 previously refractory anal fistulas. That material was the GORE° BIO-A® Fistula Plug, which is a synthetic scaffold comprising randomly arranged fibers comprising polymers of PGA and TMC. The manufacturer of GORE® BIO-A® Fistula Plug describes it as easy to use with no operative preparation, such as soaking or stretching (GORE® BIO-A® Fistula Plug, Frequently Asked Questions, September 2010).

Having the ability to select a material and then seed that selected material with adipose derived mesenchymal stem cells as described herein to create an implant that can be used to treat over 80 percent of refractory fistulas (e.g., refractory anal fistulas) successfully without future fistula recurrence provides both clinicians and patients with a long awaited treatment option for these serious medical conditions.

This document also provides methods and materials for treating wounds (e.g., non-healing wounds or abscesses). For example, this document provides methods and materials for applying a synthetic scaffold that includes fibers comprising polymers of PGA and TMC and that is seeded with mesenchymal stem cells (e.g., adipose derived mesenchymal stem cells) located in the spaces between the fibers to a wound of a mammal (e.g., a human). In some cases, a synthetic scaffold provided herein can be used to treat wounds (e.g., non-healing wounds or abscesses).

In general, one aspect of this document features a method for treating a fistula in a mammal. The method comprises (or consists essentially of or consists of) implanting a scaffold into the fistula, wherein the scaffold comprises fibers (e.g., randomly arranged fibers) and mesenchymal stem cells located between the fibers, wherein the fibers comprise polymers of polyglycolic acid and trimethylene carbonate. The mammal can be a human. The fistula can be an anal fistula. The fistula can be a refractory anal fistula. A maximum diameter of the fistula can be less than 25 mm. The mesenchymal stem cells can be adipose derived mesenchymal stem cells. The polyglycolic acid can be about 60 to about 70 percent of the fibers. The polyglycolic acid can be about 67 percent of the fibers. The trimethylene carbonate can be about 30 to about 40 percent of the fibers. The trimethylene carbonate can be about 33 percent of the fibers. The scaffold can comprise platelet derivative material.

In another aspect, this document features a method for making an implant for treating a fistula. The method comprises (or consists essentially of or consists of) contacting a scaffold comprises fibers (e.g., randomly arranged fibers) with mesenchymal stem cells within a polypropylene container, wherein the fibers comprise polymers of polyglycolic acid and trimethylene carbonate. The mesenchymal stem cells can be adipose derived mesenchymal stem cells. The contacting within the polypropylene container can occur for more than three days. The contacting within the polypropylene container can occur for from about three days to about ten days. The contacting within the polypropylene container can occur for from about four days to about six days. The polyglycolic acid can be about 60 to about 70 percent of the fibers. The polyglycolic acid can be about 67 percent of the fibers. The trimethylene carbonate can be about 30 to about 40 percent of the fibers. The trimethylene carbonate can be about 33 percent of the fibers. The method can comprise contacting the scaffold with platelet derivative material within the container.

In another aspect, this document features a scaffold comprising (or consisting essentially of or consisting of) fibers and mesenchymal stem cells located between the fibers, wherein the fibers comprise (or consist essentially of or consist of) polymers of polyglycolic acid and trimethylene carbonate, and wherein the mesenchymal stem cells express more fibroblast growth factor 2 (FGF-2) polypeptide, eotaxin polypeptide, FMS-like tyrosine kinase 3 ligand (FLT3L) polypeptide, growth-regulated protein (GRO) polypeptide, and interleukin 10 (IL-10) polypeptide than comparable mesenchymal stem cells cultured in the absence of the fibers, and wherein the mesenchymal stem cells express less fractalkine polypeptide than the comparable mesenchymal stem cells. The mesenchymal stem cells can be adipose derived mesenchymal stem cells. The polyglycolic acid can be about 60 to about 70 percent of the fibers. The polyglycolic acid can be about 67 percent of the fibers. The trimethylene carbonate can be about 30 to about 40 percent of the fibers. The trimethylene carbonate can be about 33 percent of the fibers. The scaffold can comprise platelet derivative material. The fibers can be randomly arranged fibers. The mesenchymal stem cells can express more monocyte-chemotactic protein 3 (MCP-3) polypeptide than the comparable mesenchymal stem cells. The mesenchymal stem cells can express less interleukin 12 (IL-12) p40 polypeptide than the comparable mesenchymal stem cells. The mesenchymal stem cells can express more interleukin 12 (IL-12) p70 polypeptide than the comparable mesenchymal stem cells.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is an anatomical schematic depicting various types of anal fistulas.

FIG. 2 is an illustration of an example solid matrix scaffold device for treatment of fistulas.

FIG. 3 is a flowchart of exemplary steps that can be used to make and implant a scaffold provided herein.

FIG. 4 is a photograph of culturing system for seeding scaffolds with adipose derived mesenchymal stem cells.

FIG. 5 is a graph plotting the pH of media versus time post seeding scaffolds with adipose derived mesenchymal stem cells. “B” represents biologic. Control is free floating adipose derived mesenchymal stem cells without any scaffold to attach to.

FIG. 6 is a graph plotting the pH of media versus time post seeding scaffolds with adipose derived mesenchymal stem cells. “S” represents synthetic. Control is free floating adipose derived mesenchymal stem cells without any scaffold to attach to.

FIG. 7 is a graph plotting the number of cells in the scaffold at 72 hours for the indicated scaffold material. “B” represents biologic; “S” represents synthetic. Control is free floating adipose derived mesenchymal stem cells without any scaffold to attach to. The dashed horizontal line is the number of cells that were seeded at hour zero onto each material (i.e., 250, 000). At 72 hours after seeding, scaffolds were collected, and quantative DNA analysis was performed to determine the number of cells in each scaffold.

FIG. 8 is a graph plotting the signal intensity for VEGF from the cells seeded into the indicated scaffolds.

FIG. 9 is a graph plotting the signal intensity for MIP-1a from the cells seeded into the indicated scaffolds. FIG. 10 is a graph plotting the signal intensity for MCP-1 from the cells seeded into the indicated scaffolds.

FIG. 11 is a graph plotting the signal intensity for EGF from the cells seeded into the indicated scaffolds.

FIGS. 12A-B. Clinical improvement of fistulizing disease after treatment with MSC bound matrix. Pre- and post-treatment (seven months after plug placement) imaging in an exemplar patient on study (A). Arrow indicates intersphincteric fistula with seton at MR imaging in 39 year-old female Crohn's patient prior to treatment and six months after therapy, along with images from perianal examination at time of plug placement (top row) and follow-up MRI. (B) Cumulative results of the changes in Van Aasche scale, tract length and fistula diameter. P values represent paired T test before and six months after plug placement. For the fistula diameter, the P value on the upper is representative of the all of the samples while the P value below is for the 11 samples with a starting diameter less than 20 mm.

FIGS. 13A-B. Altered and consistent gene expression changes after binding human mesenchymal stromal cells to polyglycolic acid trimethylene carbonate matrix. Six human adipose samples from patients with fistulizing Crohn's disease were used to expand mesenchymal stromal cells. Cells were expanded and used directly or bound to polyglycolic acid trimethylene carbonate based artificial matrix. (A) Expression values of representative genes from RNA-SEQ data. (B) Representative genes that can be used to identify the changes associated with the transition of cells after attachment to matrix.

FIGS. 14A-B. MSCs bound to matrix reduced proliferation and cell cycle, maintain secreted protein and increase matrix gene expression profiles. Top 25 highest differentially expressed genes in MSCs versus MSCs cultured on matrix (A) and genes with the highest differential expression after adherence to matrix (B). The distribution and nature of the genes identified suggest a cells on the matrix appear to have reached a post-proliferative state and exhibit increased expression of genes required for the protein synthesis machinery matrix expression. The latter facilitates a protein anabolic state that supports production of a collagen-rich extracellular matrix (ECM). Based on our mRNA analysis, this ECM is predicted to be composed of collagens types I, III, VI and V, respectively, in order of abundance.

FIGS. 15A-D. Preparation and characterization of MSC bound fistula plug for treatment of fistulizing disease in Crohn's patients. Adipose tissue from Crohn's patients was used to isolate and prepare MSC. Cells from patients (n=7) grew rapidly, recovered from frozen storage and bound with high efficiency to the matrix (A). Representative phenotype of patient MSC (B). Cell morphology at time of collection and example of prepared cell/matrix combination prior to administration (C). (D) Demonstration of viable cells (green) after binding to MSC (upper left; Syto13 positive Ethidium bromide negative), collagen deposition demonstrated by Goldner's Trichrome staining (upper right) and SEM of the matrix before (bottom left) and after cell binding (bottom right).

FIGS. 16A-D are tables showing the differential secretion of polypeptide analytes from cells located on the GORE synthetic scaffold or other synthetic materials as indicated as compared to control cells in culture media.

FIGS. 17A-D are tables showing the differential secretion of polypeptide analytes from cells located on the GORE synthetic scaffold or other synthetic materials as indicated as compared to control cells in culture media.

### DETAILED DESCRIPTION

This document provides methods and materials for treating fistulas (e.g., refractory fistulas such as refractory anal fistulas). For example, this document provides methods and materials for implanting a synthetic scaffold (e.g., fistula plug) comprising randomly arranged fibers comprising polymers of PGA and TMC and seeded with mesenchymal stem cells (e.g., adipose derived mesenchymal stem cells) located in the spaces between the randomly arranged fibers into a fistula (e.g., refractory anal fistula) of a mammal (e.g., a human).

A synthetic scaffold provided herein can include fibers comprising polymers of PGA and TMC that are designed or molded into any appropriate shape and dimension. For example, a synthetic scaffold provided herein can be designed or molded into a shape and dimension that conforms to a non-healing wound or fistula. Examples of appropriate shapes include, without limitation, patches, sheets, tubes, plugs, or columns. In one example, a sheet can be applied to a surface of a wound. In another example, a sheet can be rolled to form a tube-like structure to wrap around a tubular structure or to support a lumen. In some cases, a synthetic scaffold in a sheet format can be used to treat a bronchopleural fistula.

A fistula is a tunnel between two hollow organs, or between a hollow organ and the surface of the skin. Any appropriate fistula can be treated as described herein. For example, anal fistulas, enterocutaneous fistulas, bronchopleural fistulas, and vesicocutaneous fistulas can be treated as described herein. In some cases, the methods and materials provided herein can be used to treat refractory fistulas. As used herein, the term “refractory” as used with respect to fistulas refers to those fistulas that have failed to heal despite current best practice which includes medical and surgical therapy. Examples of refractory fistulas that can be treated as described herein include, without limitation, refractory anal fistulas and refractory enterocutaneous fistulas.

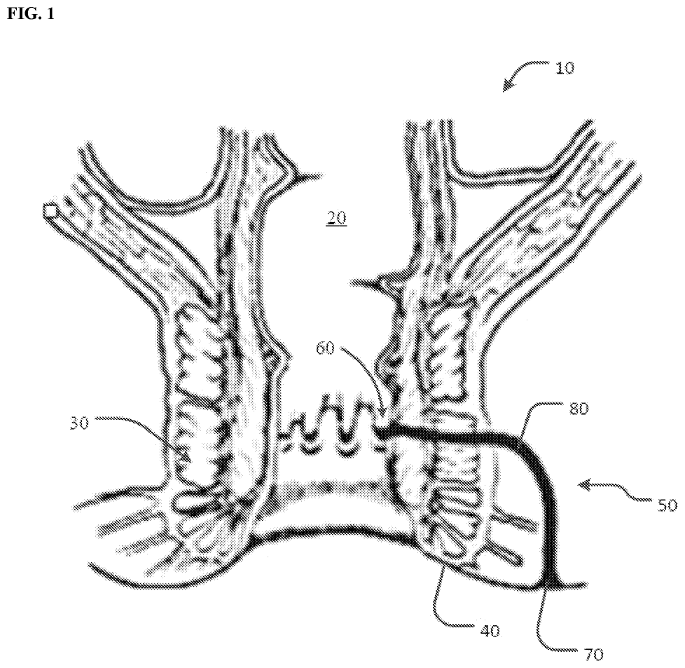

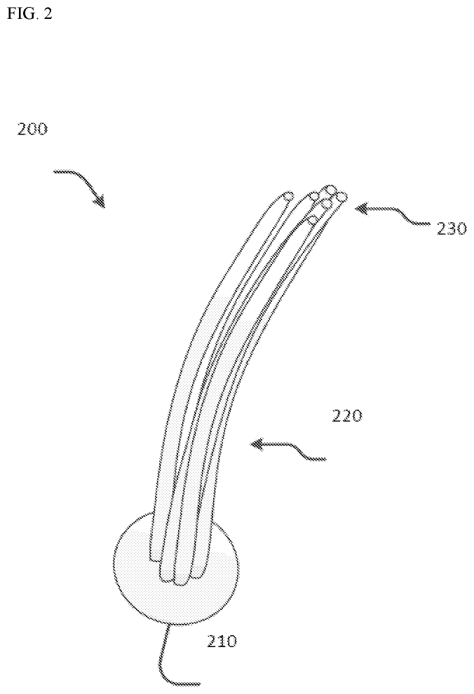

FIG. 1 provides an anatomical schematic drawing of a human's lower colon area 10. Lower colon area 10 includes rectum 20, anal sphincter muscles 30, and skin surface 40.

An anal fistula 50 also is depicted. Types of anal fistulas are classified based on the path of their tracts and how close they are to the sphincter muscles. For example, anal fistula 50 is a trans-sphinteric fistula. However, the example devices, systems, and methods provided herein can be applicable to other types of anal fistulas, and to fistulas in general. Anal fistula 50 includes an internal opening 60 (in rectum 20), an external opening 70 (on skin surface 40), and a fistula tract 80. Fistula tract 80 is a tunnel connecting internal opening 60 to external opening 70. Fistula tract 80 is an example of a type of abscess cavity. Fistula tract 80 can be treated by the devices, systems, and methods provided herein. Other types of fistulas can be similarly treated.

FIG. 2 depicts an example embodiment of a fistula repair device 200 (e.g., a fistula plug) for treating an anal fistula, such as anal fistula 50 of FIG. 1. Fistula repair device 200 is an example of an implantable bioabsorbable device that provides a solid matrix scaffold to support tissue growth. Devices, such as fistula repair device 200 with a solid matrix scaffold, can be implanted into fistulas to facilitate tissue regeneration and healing of the cavity. For example, cells can migrate into the solid matrix scaffold, and tissue can be generated as the body gradually absorbs the solid matrix scaffold material.

A synthetic scaffold (e.g., fistula plug) provided herein such as fistula repair device 200 can include randomly arranged fibers comprising polymers of PGA and TMC. Any appropriate amount of PGA and TMC can be used to make such synthetic scaffolds. For example, a synthetic scaffold (e.g., fistula plug) provided herein can include from about 50 percent to about 80 percent (from about 55 percent to about 80 percent, from about 60 percent to about 80 percent, from about 50 percent to about 70 percent, or from about 65 percent to about 70 percent) of PGA and from about 20 percent to about 50 percent (from about 25 percent to about 50 percent, from about 30 percent to about 50 percent, from about 20 percent to about 40 percent, or from about 30 percent to about 35 percent) of TMC. In some cases, a synthetic scaffold (e.g., fistula plug) provided herein can include about 67 percent of PGA and about 33 percent TMC. One example of a synthetic scaffold that can be used as described herein is the GORE® BIO-A® Fistula Plug.

As described herein, solid matrix scaffold devices, such as example fistula repair device 200, can be impregnated with mesenchymal stem cells (e.g., adipose derived mesenchymal stem cells) to create an improved implantable device to treat fistulas (e.g., refractory anal fistulas) with a greater than 80 percent success rate. For example, a synthetic scaffold (e.g., fistula plug) provided herein such as fistula repair device 200 having randomly arranged fibers comprising polymers of PGA and TMC can be seeded with mesenchymal stem cells (e.g., adipose derived mesenchymal stem cells) that become located in the spaces between the randomly arranged fibers.

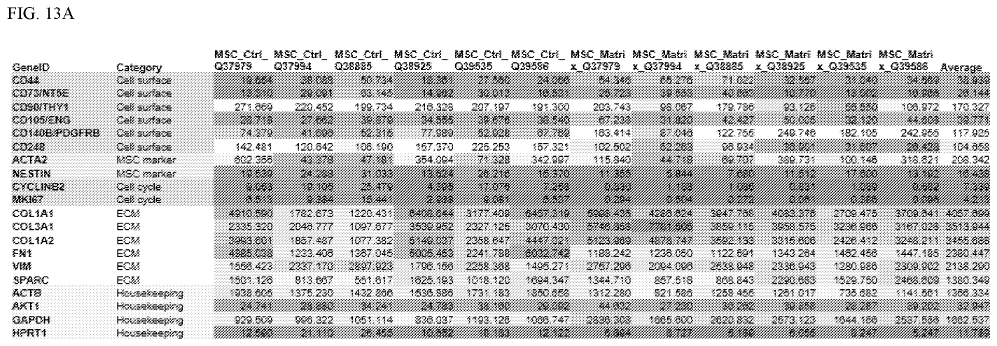

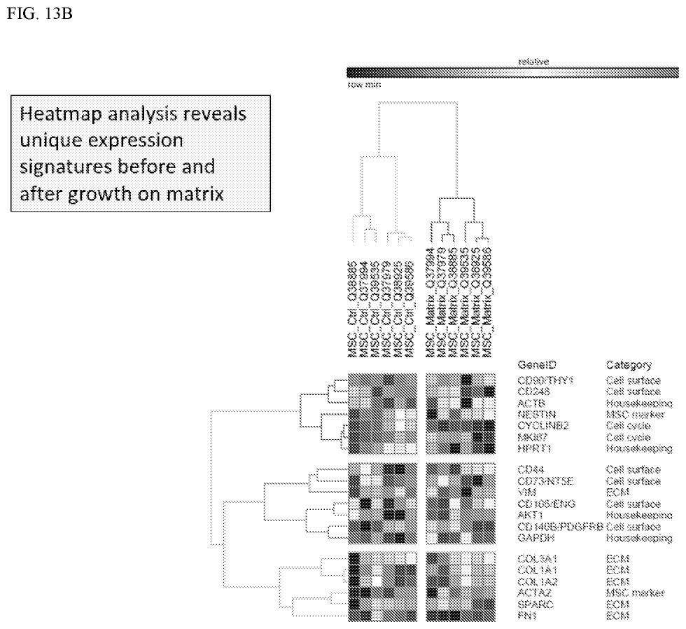

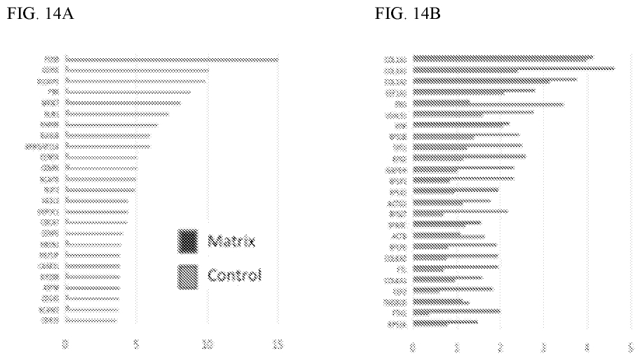

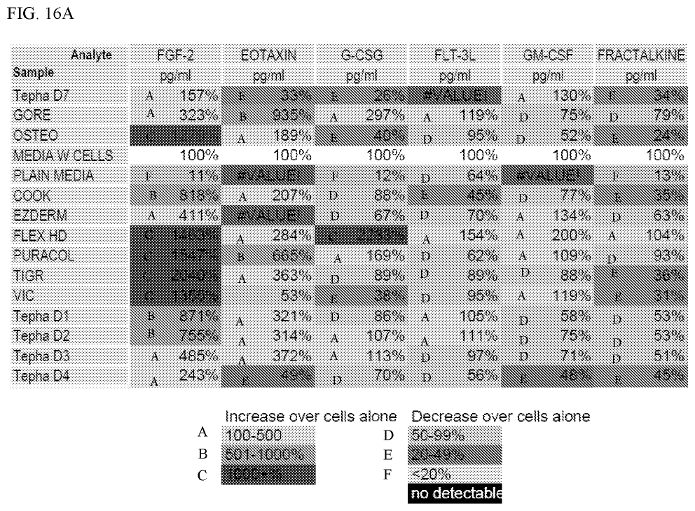

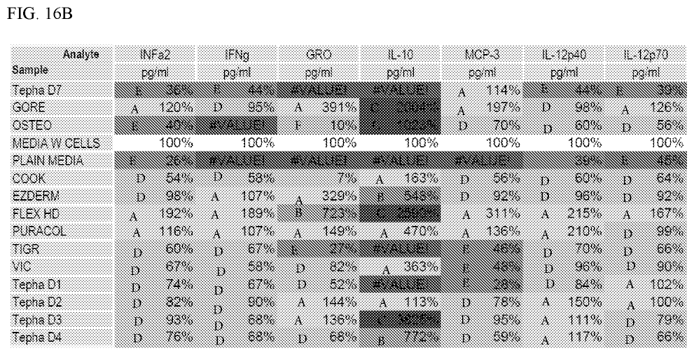

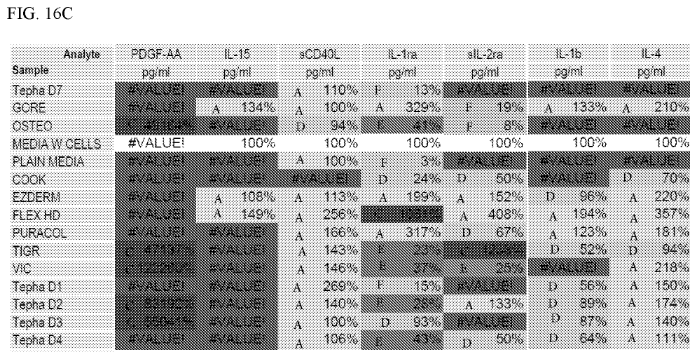

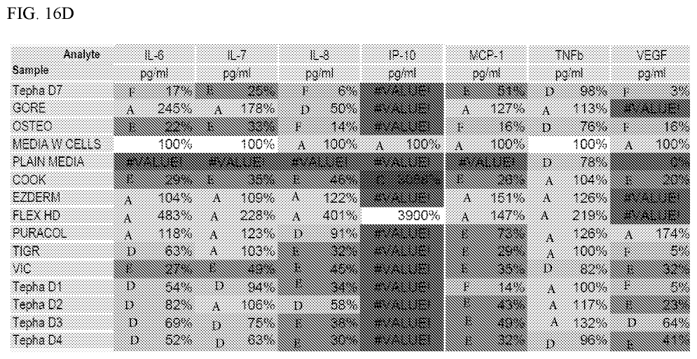

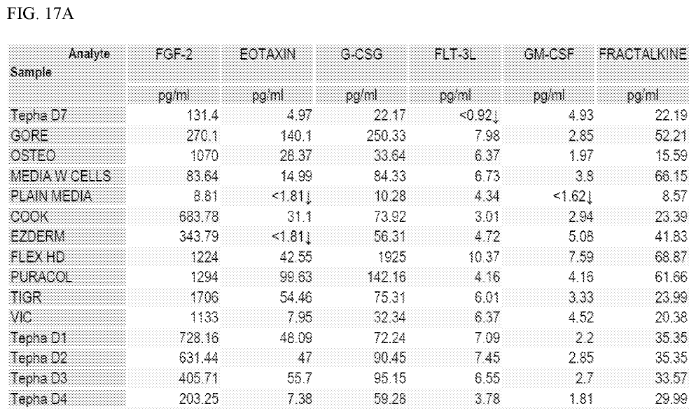

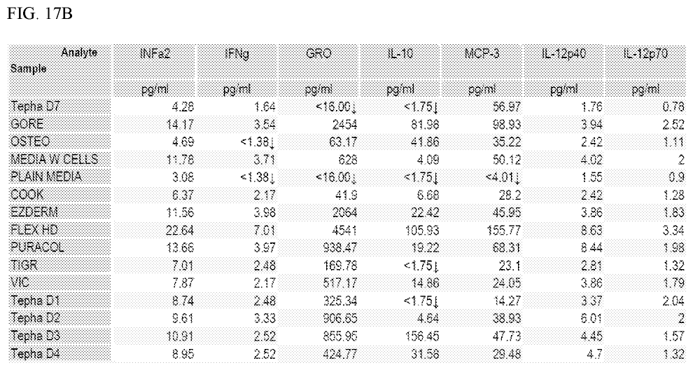

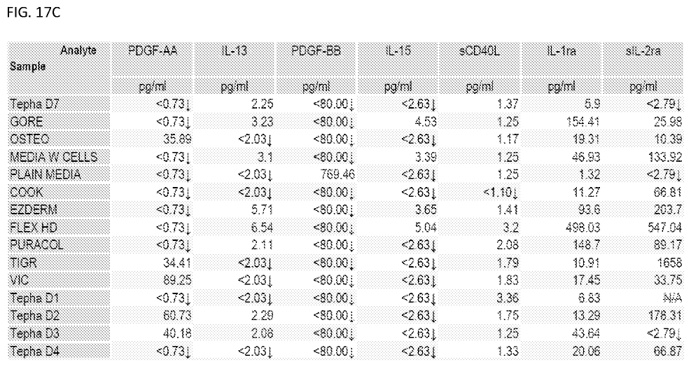

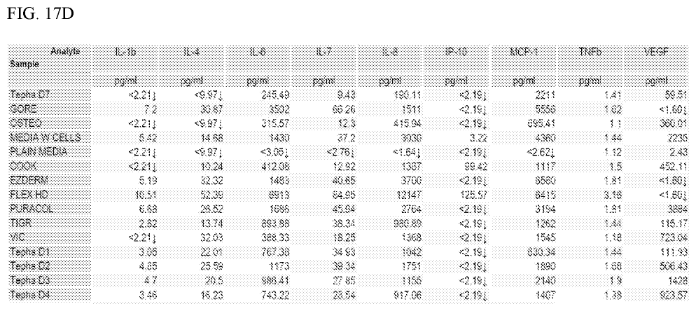

In some cases, a synthetic scaffold comprising fibers (e.g., randomly arranged fibers) comprising polymers of PGA and TMC can be designed to include mesenchymal stem cells (e.g., adipose derived mesenchymal stem cells) located in the spaces between the fibers (e.g., randomly arranged fibers) wherein the cells have a unique polypeptide expression profile. For example, the cells of the synthetic scaffold can express one or more (e.g., 1 to 10, 1 to 15, 5 to 10, 5 to 15, 10 to 15, 15 to 20, 20 to 25, 25 to 30, or 30-35) of the polypeptides listed in FIG. 13A or FIG. 13B in a manner as shown in FIG. 13A or FIG. 13B under a “matrix” column, as compared to a “ctrl” (control) column, or listed in FIG. 16 or FIG. 17 in a manner as shown in FIG. 16 or FIG. 17 that demonstrated differential secretion of analyte from cells located on the GORE synthetic scaffold compared to control cells in culture media. In some cases, a synthetic scaffold comprising fibers (e.g., randomly arranged fibers) comprising polymers of PGA and TMC can be designed to include mesenchymal stem cells (e.g., adipose derived mesenchymal stem cells) located in the spaces between the fibers (e.g., randomly arranged fibers) wherein the cells express more CD44, CD105/ENG, AKT1, CD140B/PDGFRB, GAPDH, and/or COL3A1 polypeptides (and/or less CD90/THY1, CD248, ACTB, Nestin, CyclinB2, MKI67, and/or HPRT1 polypeptides) than that observed in a random collection of control mesenchymal stem cells (e.g., adipose derived mesenchymal stem cells) not contacted with the synthetic scaffold. In some cases, a synthetic scaffold comprising fibers (e.g., randomly arranged fibers) comprising polymers of PGA and TMC can be designed to include mesenchymal stem cells (e.g., adipose derived mesenchymal stem cells) located in the spaces between the fibers (e.g., randomly arranged fibers) wherein the cells exhibit higher RNA expression of COL1A1, COL1A2, VIM, CD140B/PDGFRB, and/or COL3A1 (and/or exhibit lower RNA expression of CD90/THY1, CD73, CD248, ACTB, Nestin, CyclinB2, MKI67, and/or HPRT1) than that observed in a random collection of control mesenchymal stem cells (e.g., adipose derived mesenchymal stem cells) not contacted with the synthetic scaffold. In some cases, a synthetic scaffold including fibers comprising polymers of PGA and TMC can be designed to include mesenchymal stem cells (e.g., adipose derived mesenchymal stem cells) located in the spaces between the fibers wherein the cells secreted at a higher rate the following polypeptides: FGF2, Eotaxin, G-CSG, GRO, IL-1ra, and/or IL-10 (and/or at a lower secretion rate for Fractalkine or sIL-2ra) than control cells not on the synthetic scaffold.

In some cases, the mesenchymal stem cells (e.g., adipose derived mesenchymal stem cells) used to make an implantable device as described herein can be autologous to the mammal (e.g., human) being treated. For example, a fat tissue sample can be obtained from a mammal (e.g., a human) to be treated. That obtained fat tissue sample can be processed as described elsewhere (Bartunek et al., Cell Transplantation, 20(6):797-811 (2011) and Chen et al., Transfusion, 55(5):1013-1020 (2015)), and the resulting material expanded in culture to obtain a culture of mesenchymal stem cells (e.g., adipose derived mesenchymal stem cells). In some cases, the mesenchymal stem cells (e.g., adipose derived mesenchymal stem cells) can be expanded in culture from about 3 days to about 30 days (e.g., from about 3 days to about 25 days, from about 3 days to about 15 days, from about 5 days to about 30 days, from about 10 days to about 30 days, from about 5 days to about 21 days, or from about 8 days to about 15 days). In some cases, allogeneic or xenogeneic mesenchymal stem cells (e.g., adipose derived mesenchymal stem cells) can be used instead of autologous cells.

Any appropriate method can be used to seed mesenchymal stem cells (e.g., adipose derived mesenchymal stem cells) into a scaffold having randomly arranged fibers comprising polymers of PGA and TMC. For example, a scaffold having randomly arranged fibers comprising polymers of PGA and TMC (e.g., a GORE® BIO-A® Fistula Plug) can be combined with an appropriate number of viable mesenchymal stem cells (e.g., viable adipose derived mesenchymal stem cells) in a polypropylene or polypropylene-coated container along with an appropriate media for a period of time. Any appropriate polypropylene or polypropylene-coated container can be used such as polypropylene-coated tubes, polypropylene-coated dishes, or polypropylene-coated plates. In general, from about 50,000 to about 4,000,000 (e.g., from about 100,000 to about 4,000,000, from about 200,000 to about 4,000,000, from about 250,000 to about 4,000,000, from about 200,000 to about 3,500,000, from about 200,000 to about 3,000,000, from about 200,000 to about 2,500,000, or from about 250,000 to about 3,000,000) viable mesenchymal stem cells (e.g., viable adipose derived mesenchymal stem cells) per cm² of scaffold material can be used to seed the scaffold. Examples of media that can be used to seed a scaffold as described herein include, without limitation, aMEM, DMEM, RPMI, Eagles MEM, ADSC, MSCGM, and specialty MSC media growth products. These media may or may not include media supplements consisting of derivatives of human platelet lysate such as PLTMax® (Mill Creek Life Sciences, LLC; Rochester, Minn.). In general, the seeding process can be from about 1 day to about 10 days (e.g., from about 2 days to about 10 days, from about 3 days to about 10 days, from about 1 day to about 8 days, from about 1 day to about 6 days, from about 3 days to about 6 days, or from about 4 days to about 6 days). After culturing the scaffold with mesenchymal stem cells (e.g., viable adipose derived mesenchymal stem cells) to seed the scaffold with cells, the seeded scaffold can be implanted into the mammal (e.g., human) to treat the fistula.

In some cases, one or more therapeutic agents can be combined with a scaffold provided herein via, for example, appropriate covalent or non-covalent binding. Example of therapeutic agents that can be combined with a scaffold provided herein include, without limitation, growth factors such as PDGF, FGF, or VEGF and platelet material such as pooled human platelet derivatives or platelet lysate material. A process of binding therapeutic agents to a solid matrix scaffold provided herein can be performed, in some embodiments, by suspending the therapeutic agents in various types of solutions or materials that can then be combined with the scaffold material to imbibe the scaffold material with the therapeutic agent. In some cases, one or more therapeutic agents can be covalently or non-covalently bound to the scaffold material during the cell seeding process. In some cases, a scaffold such as fistula repair device 200 can be soaked in a solution containing mesenchymal stem cells (e.g., adipose derived mesenchymal stem cells) alone or mesenchymal stem cells (e.g., adipose derived mesenchymal stem cells) and platelet lysate material in suspension.

Referring to FIG. 2, in general, example fistula repair device 200 (e.g., a fistula plug device) can include a disk portion 210 and multiple legs 220. The multiple legs 220 can be attached to disk portion 210 on their proximal ends, while distal ends 230 can be unattached and individually free. The multiple legs 220 can provide a fistula repair device 200 that is customizable to fit various sizes of fistula tracts. That is, one or more of multiple legs 220 can be trimmed from the disk portion 210 in order to reduce the cross-sectional size of fistula repair device 200 to correlate with the size of the particular fistula tract being treated.

Other embodiments of fistula repair devices provided herein can have a variety of different physical configurations. For example, in some cases, a fistula repair device can be a single elongate element with an elongated conical shape. Further, in some cases, the fistula repair device can be a single element with an elongated cylindrical shape. In some embodiments, the fistula repair device can have a variable profile along the length of the device. In general, the fistula repair device can be shaped to fill the cavity and to remain securely implanted. In some cases, a fistula repair device provided herein can be a sheet placed over one or both ends of the fistula. The fistula repair devices, as described herein, can be made from synthetic polymers of PGA and TMC or from a composite construction of such materials.

The example fistula repair device 200 with seeded mesenchymal stem cells (e.g., adipose derived mesenchymal stem cells) (and/or platelet lysate material) can be implanted in the tract of a fistula according to the following general exemplary process. First, distal ends 230 can be sutured together. A suitable pulling device can be inserted all the way through fistula tract 80 (refer also to FIG. 1). The pulling device can be a suture, guidewire, hemostat, and the like, in accordance with the particular anatomy and type of the fistula being treated. The end of the pulling device at internal opening 60 can be attached to distal ends 230 of fistula repair device 200. For example, in the case of a suture pulling device, the suture pulling device can be stitched and/or tied to distal ends 230. Or, in the case of a hemostat pulling device, the hemostat can be clamped to distal ends 230. Next, the other end of the pulling device at external opening 70 can be carefully pulled to draw distal ends 230 towards internal opening 60. As distal ends 230 approach internal opening 60, distal ends 230 can be carefully guided into fistula tract 80 through internal opening 60. Fistula repair device 200 can be pulled all the way into fistula 50 until disk portion 210 is flush with internal opening 60. Disk portion 210 can then be sutured or clamped to secure it in place at internal opening 60. If distal ends 230 are protruding from external opening 70, they can be trimmed flush to skin surface 40.

The implanted fistula repair device 200 seeded with mesenchymal stem cells (e.g., adipose derived mesenchymal stem cells) can provide a scaffold for soft tissue repair to thereby facilitate healing and closure of the fistula. Combining a scaffold comprising randomly arranged fibers comprising polymers of PGA and TMC with seeded mesenchymal stem cells (e.g., adipose derived mesenchymal stem cells) that become located in the spaces between the randomly arranged fibers can result in a device that can achieve improved fistula treatment success as compared to other devices made from materials other than polymers of PGA and TMC. That improved fistula treatment success can be greater than 80 percent when treating refractory fistulas such as refractory anal fistulas.

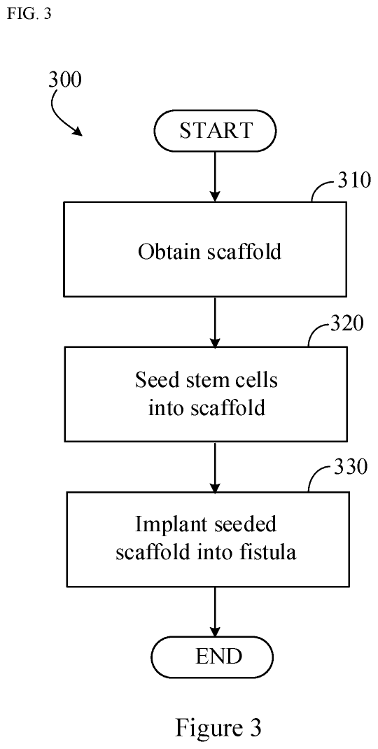

FIG. 3 is a flowchart depicting an example process 300 for treating a fistula using a system including a scaffold containing mesenchymal stem cells (e.g., adipose derived mesenchymal stem cells). In general, the technique of example process 300 includes filling fistula with a scaffold that comprises randomly arranged fibers comprising polymers of PGA and TMC and mesenchymal stem cells (e.g., adipose derived mesenchymal stem cells).

At step 310, a scaffold comprising randomly arranged fibers comprising polymers of PGA and TMC is obtained. The scaffold can be a GORE® BIO-A® Fistula Plug. Before step 320, stem cells can be obtained. For example, adipose derived mesenchymal stem cells can be obtained from a mammal (e.g., a human) being treated. At step 320, the scaffold obtained at step 310 can be contacted with adipose derived stem cells (e.g., adipose derived mesenchymal stem cells) to seed the scaffold with the cells. In some cases, mesenchymal stem cells (e.g., adipose derived mesenchymal stem cells) can be autologous, i.e., derived from the patient to be treated with the scaffold. In some cases, mesenchymal stem cells may require culturing and processing according to established protocols for providing control of the process. For example, mesenchymal stem cells for clinical use may require ex vivo expansion of the mesenchymal stem cells in media containing supplements such as fetal bovine serum or, alternatively, human platelet derivatives or human platelet lysate material. At step 320, techniques for processing and culturing the cells can be performed, or the cells can otherwise be obtained.

In some cases, a solution for seeding the scaffold with mesenchymal stem cells (e.g., adipose derived mesenchymal stem cells) can be designed to include (in addition to the cells) components including, without limitation, platelet derivatives (e.g., human platelet derivatives), platelet lysate material (e.g., human platelet lysate material), salts, buffers, growth factors, cell signaling agents, or small molecule modulators. In these cases, a scaffold material can be soaked in the solution, or imbibed with the solution using another suitable technique.

In one example, when using platelet derivatives (e.g., human platelet derivatives) or platelet lysate material (e.g., human platelet lysate material), the scaffold material can be soaked in a solution containing the platelet derivatives (e.g., human platelet derivatives) or the platelet lysate material (e.g., human platelet lysate material) for a range of time from about 3 minutes to about 5 days (e.g., from about 5 minutes to about 5 days, from about 15 minutes to about 5 days, from about 1 hour to about 5 days, from about 3 hours to about 5 days, from about 6 hours to about 5 days, from about 18 hours to about 5 days, from about 1 day to about 5 days, from about 2 days to about 5 days, from about 3 days to about 5 days, or from about 4 days to about 5 days). In some cases, a range of time from about 3 minutes to about 4 days (e.g., from about 3 minutes to about 3 days, from about 3 minutes to about 2 days, from about 3 minutes to about 1 day, from about 3 minutes to about 12 hours, from about 3 minutes to about 6 hours, from about 3 minutes to about 4 hours, or from about 3 minutes to about 2 hours) can be used, or a range of time from about 1 hour to about 3 days (e.g., from about 2 hours to about 2 days, from about 2 hours to about 1 day, or from about 1 day to about 3 days) can be used.

The soaking step can be performed at any appropriate temperature. In one example, the soaking step can be performed at a range of temperatures from about 2° C. to about 45° C. (e.g., from about 10° C. to about 40° C., from about 20° C. to about 37° C., or from about 30° C. to about 40° C.). In another example, the soaking step can be performed at a range of temperatures from about 18° C. to about 26° C. (e.g., from about 20° C. to about 24° C. or from about 21° C. to about 23° C.). In another example, the soaking step can be performed at a range of temperatures from about 30° C. to about 44° C. (e.g., from about 33° C. to about 41° C. or from about 36° C. to about 38° C.). In another example, the soaking step can be performed at a range of temperatures from about 1° C. to about 7° C. (e.g., from about 3° C. to about 5° C.).

In another example, a solid matrix scaffold material can be soaked in a solution (e.g., a platelet lysate material-containing solution) for about 24 hours at about 37° C.

At step 330, the solid matrix scaffold seeded with mesenchymal stem cells (e.g., adipose derived mesenchymal stem cells) is implanted into a fistula (e.g., a refractory fistula such as a refractory anal fistula) being treated. With the system in place in the fistula, the solid matrix scaffold can promote tissue growth and healing of the fistula.

The invention will be further described in the following examples, which do not limit the scope of the invention described in the claims.

### EXAMPLES

### Example 1—Assessing Scaffolds

Ten FDA approved materials were selected from commercially available matrices and tested in vitro: FlexHD (Biologic; Decellularized Human Dermis; Musculoskeletal Transplant Foundation), PuraCol (Biologic; Purified Type 1 Bovine Tendon Collagen; Medline Industries Inc.), EZ Derm (Biologic; Aldehyde Crosslinked Decellularized Porcine Dermis; Molnlycke Inc.), Cook SIS (Biologic), Gore Bio-A (Synthetic), Osteopore (Synthetic; 3D Printed Polycarpolactone; Osteopore International Pte Ltd.), Gore TRM (Synthetic), Tepha-P4HB (Synthetic; Poly-4 Hydroxybutyrate; Bacterial Bioplastic; Tepha Corporation), TIGR Matrix (Synthetic; Mesh of Polyglycolic Acid, Polylactic Acid, and Trimethylene Carbonate; Novus Scientific), and Vicryl 910 (Synthetic; PLGA; Ethicon Inc.)

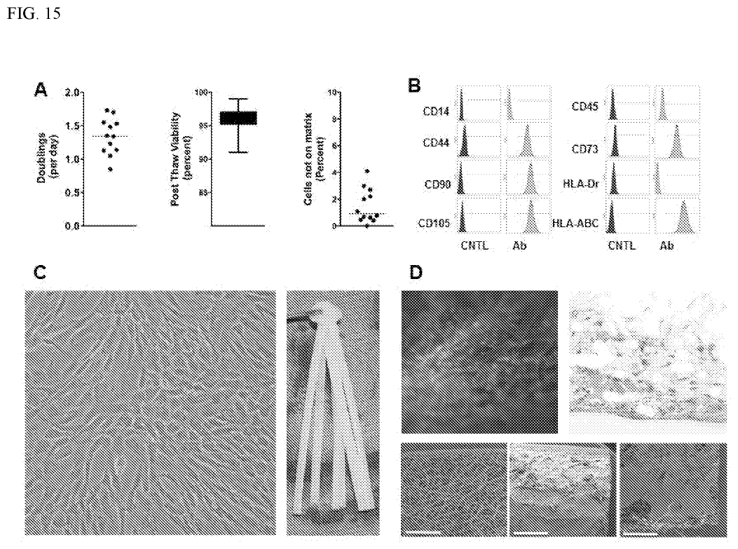

The Gore Bio-A Plug was an electrospun synthetic plug made from polymers of PGA:TMC (FIG. 15D, bottom left SEM). The plug is highly porous, and the fibers are randomly aligned. The Gore TRM was an electrospun synthetic sheet made from polymers of PGA:TMC. Structurally this material is more densely packed with fibers than the Gore plug. It also is much thicker than the plug, and is clinically used for abdominal reinforcement. The Tepha P4HB is a plastic mesh made from poly-4-hydroxybutyrate (P4HB). Fibers are woven together to form large pores. P4HB is bioabsorbable over several months. Originally, this plastic, which is derived from bacteria, was to be used for biodegradable credit cards but the material was re-purposed for medical use due to its properties. It is used clinically for reinforcement applications similar to Gore TRM. Osteopore is a 3D printed scaffold made from polycarpolactone (PCL) and used mainly for joint/cartilage repair. The TIGR Matrix is an abdominal reinforcement mesh made from a combination of PGA, polylactic acid (PLA), and TMC. The materials are woven together to form a macroporous mesh. Thinner fibers dissolve over weeks, and thicker fibers dissolve over months in vivo. Vicryl 910 is an abdominal reinforcement mesh made from polyglycolic-co-lactic acid (PLGA). This material is used extensively for reinforcement. Vicryl-910 is woven and has a much smaller pore size compared to Tepha-P4HB, Osteopore, and TIGR Matrix. PLGA is absorbed by hydrolysis over the course of several weeks to months in vivo.

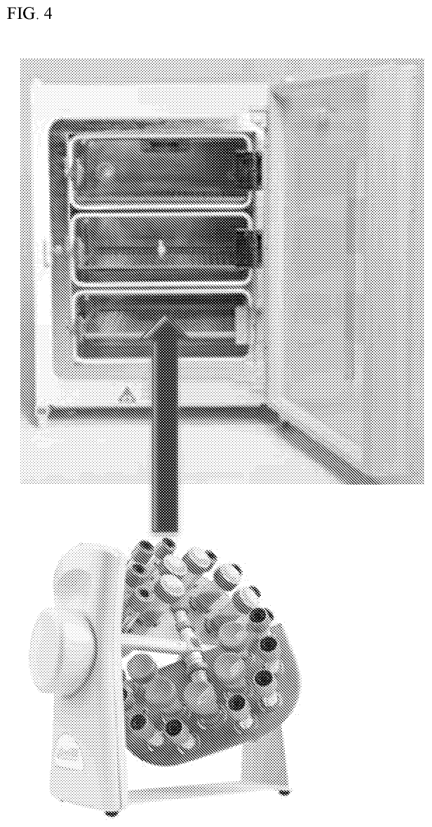

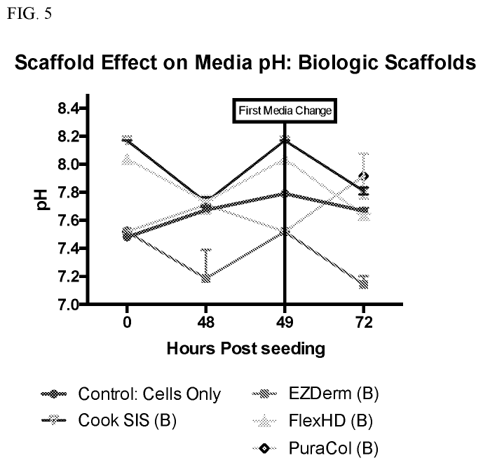

Eight 1 cm×1 cm scaffolds of each material were loaded into 15 cc polyethylene culture tubes with 250,000 adipose derived mesenchymal stem cells and culture media (a-MEM with 5% platelet lysate). Scaffolds were free-floating and rotating in an incubator for 72 hours allowing for dynamic cellular seeding (FIG. 4). Data was collected to determine the top performing matrices to be used in animal models based on scaffold effect on culture media, scaffold cellular adherence, and post adhesion cellular cytokine release.

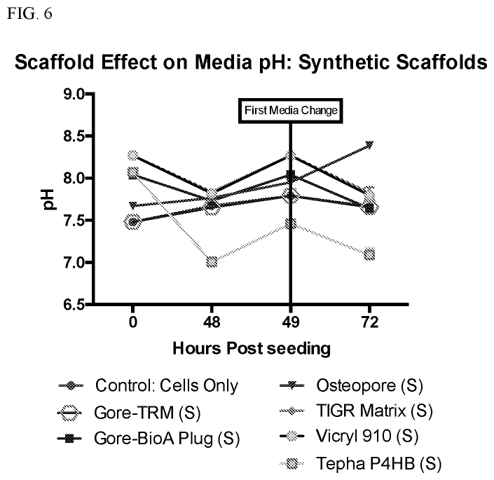

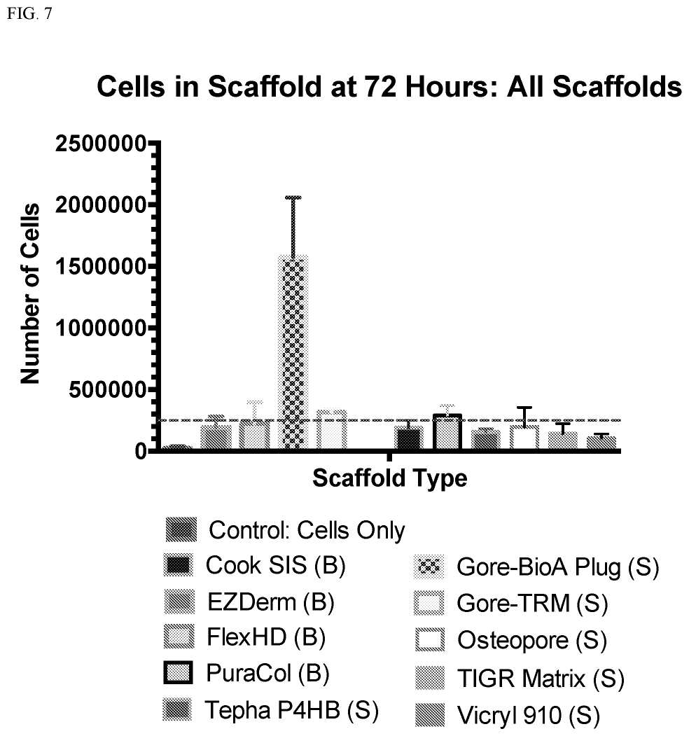

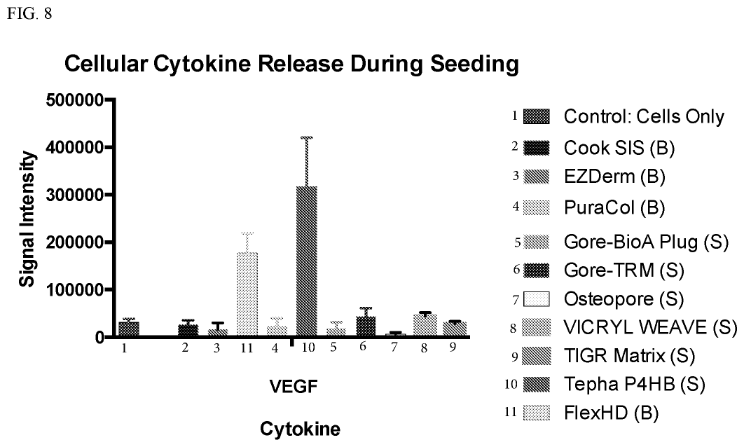

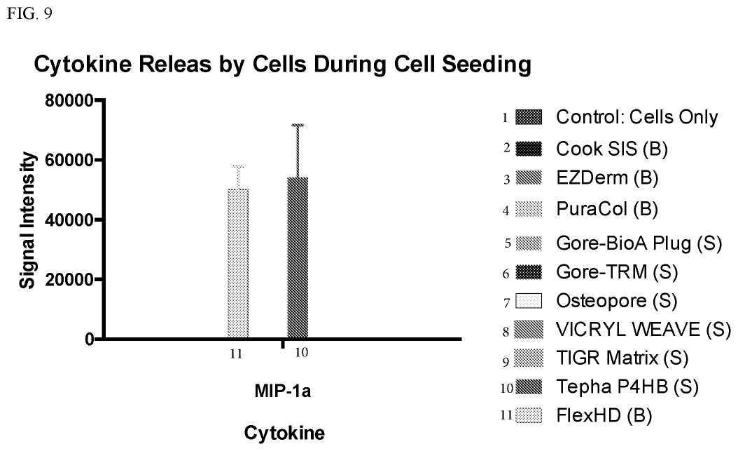

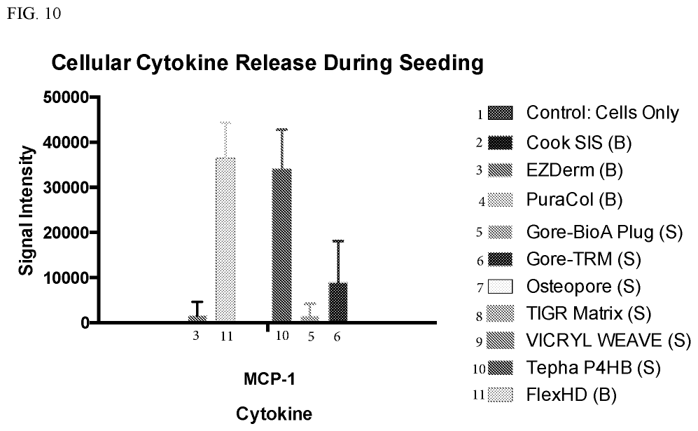

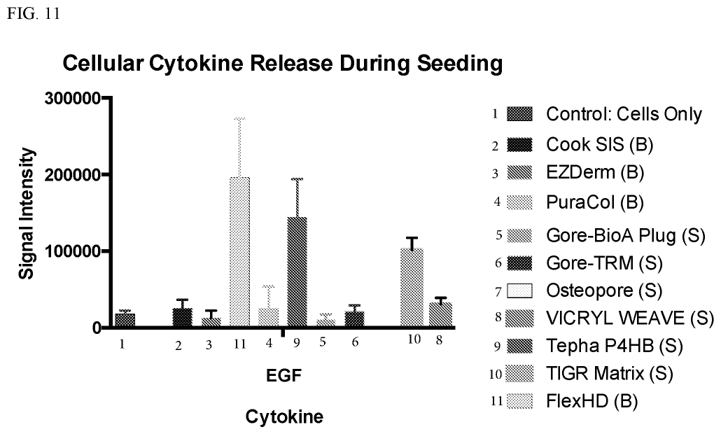

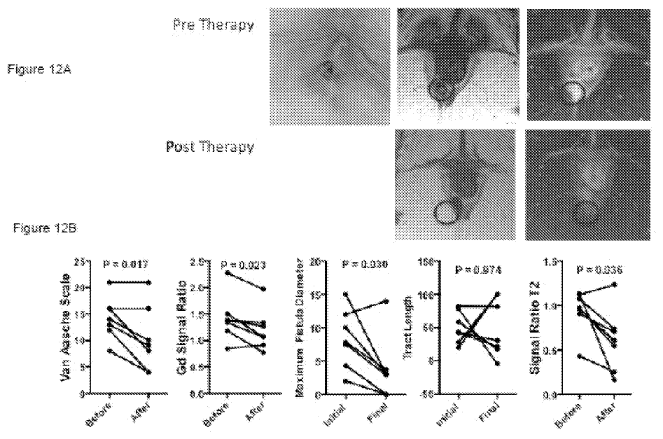

Some differences in media pH were observed (FIGS. 5 and 6). The Gore TRM, PuraCol, and FlexHD exhibited some effective seeding of adipose derived mesenchymal stem cells, but the Gore BioA plug exhibited substantial seeding and proliferation of adipose derived mesenchymal stem cells (FIG. 7). In fact, about five times more viable cells were present within the Gore BioA plug than the starting amount of adipose derived mesenchymal stem cells (i.e., 250,000). The cells from the FlexHD, Gore TRM, and Tepha P4HB scaffolds exhibited a strong angiogenic chemokine release effect (FIGS. 8-11).

### Example 2—Stem Cells on Matrix Plugs Heals Crohn's Related Perianal Fistulas

### Product Manufacturing and Trial Enrollment

Patients with Crohn's disease, ages 18-65, with a single draining fistula for at least three months despite medical therapy, without contraindication to Magnetic Resonance (MR) evaluation, and who failed standard therapy including anti-TNF therapy were eligible. Patients were excluded if they had clinically significant comorbidities within six months of MSC harvest, history of cancer, hepatitis or HIV, or were pregnant or lactating. Informed consent was obtained for all patients.

Patients underwent a baseline general exam, and serologic studies including complete blood count (CBC) with differential, C reactive protein (CRP), erythrocyte sedimentation rate (ESR), and electrolytes. Patients were scheduled for an exam under anesthesia (EUA) to confirm the fistula tract and architecture, to drain sepsis if present, and to place a seton. At the time of this operation, a 2 cm transverse incision was made in the abdominal wall to obtain up to 4 grams of adipose tissue collected under sterile conditions. After obtaining sufficient cells to harvest and load the matrix, cells were cryo-preserved, and samples were used for release testing consisting of phenotype (CD44, CD73, CD105, Class I, CD14, CD45 and Class II), mycoplasma, culture sterility (aerobic and anaerobic), and cytogenetic analysis (FIGS. 15A-B). When the patient was scheduled for plug placement, the MSCs meeting release criteria were thawed and returned to culture in the presence of a Gore® Bio-A® Fistula Plug in a polypropylene coated bioreactor for 3-6 days. Post thaw viability was calculated using trypan blue exclusion. Cell retention after cell administration to the plug was calculated by removing a sample of the supernatant, counting the cells, multiplying by the volume of media, and then expressed as a percentage of the cells delivered to the bioreactor (FIG. 15A).

Prior to administration to the patient, the media used to incubate the cells/plug combination was evaluated with a gram stain, and a sample was sent for additional sterility testing. The plug was washed to remove unbound cells and media, and then maintained in lactated ringers until delivery for administration.

Patients underwent intraoperative placement of the stem cell-loaded plug (MSC-MATRIX) approximately six weeks following the MSC harvest. The operation involved removal of the previously placed seton, curetting the fistula tract, and placement of the MSC-MATRIX fistula plug. The plug was passed through the tract and secured at the internal opening using 4 to 6 sutures. The external opening was widened appropriately to allow adequate drainage. Patients were observed for six hours for acute adverse events before discharge from the hospital, and seen again in clinic the following day. Subsequent visits occurred at week 2, and 1, 2, 3 and 6 months following MSC-MATRIX placement at which time a clinical exam was performed to (a) assess the opening of the fistula tract and (b) attempt to express any fluid from the fistula tract with deep palpation. MRI was performed prior to surgery and at 3 and 6 months.

Conventional multiplanar, multisequence pelvic MRI using a torso-phased array coil was used for perianal fistula detection and characterization. A GI radiologist with experience in interpreting pelvic MRI interpreted MRI images, classifying fistulas according to the Park's and St. James classification systems. Fistula activity was characterized using the Van Assche score, which grades fistula activity according complexity, extension, T2 hyper-intensity, and other complications (Van Assche et al., Am. J. Gastroent., 98:332-339 (2003)). Surrogate quantitative markers of fistula activity also were measured, including maximum fistula diameter and length of the hyperintense T2 tract. The length and diameter of T2-weighted hyperintensity within the fistula tract was chosen for measurement as T2-weighted hyperintensity within fistulas reflects fluid and granulation tissue, and decrease in fistula size and reduction is associated with fistula healing.

### Evaluation of Response to Treatment

### Primary Endpoint (Safety):

The primary endpoint of this study was to determine the safety and feasibility of using adipose derived, autologous mesenchymal stromal cells (MSC) bound to the Gore® Bio-A® Fistula Plug for treatment of refractory perianal fistulas. The subjects were monitored for the following adverse events:

1. Worsening (change in nature, severity, or frequency) of Crohn's disease present at the time of the study.

2. Intercurrent illnesses

3. Abnormal laboratory values (this included clinically significant shifts from baseline within the range of normal that the investigator considers to be clinically significant).

4. Clinically significant abnormalities in physical examination, vital signs, weight, drainage for the perianal fistulae.

### Secondary Endpoint (Efficacy):

A clinical assessment of drainage was performed on physical exam at the week 24 (six month) visit. Fistula closure was defined as the absence of drainage; spontaneous or with gentle compression. Radiographic response by MRI, the gold standard test for assessment of presence and activity (Gecse et al., Gut, 63:1381-1392 (2014)), was performed.

For the purposes of this study, fistula activity was defined in two ways: clinically and radiographically. Clinically, a partial response was defined as decreased drainage and symptoms, and a complete response was defined as complete cessation of drainage (some patients had a persistent skin defect preventing the use of the term “complete closure”). Radiographic response was defined by decrease in the diameter and length of the T2-weighted hyperintense fistula tract on T2-weighted fast spin-echo images (expressed as percentage change from baseline), without development of abscess or additional ramifications off the treated fistula, and without change in the Van Aasche MRI perianal fistula severity score. A decrease in the Van Aasche score was not required for treatment response, as marked reductions in fistula size can be seen without changes in the Van Aasche score; however, any increase in the Van Aasche score was considered failure of response, as an increase in fistula ramifications or abscess would increase score components.

### High Throughput RNA-Sequencing and Bioinformatic Analysis

Samples of cells from the first six patients enrolled were expanded using protocols identical to the standard operating procedures used to generate MSCs for the clinical protocol. Briefly, adipose tissue obtained at the time of surgery was transferred to a cGMP manufacturing facility. MSCs were harvested from the stromal vascular fraction of adipose tissue. The resulting MSCs were expanded ex vivo using approved protocols under cGMP conditions. Briefly, adipose tissue was washed in D-PBS, centrifuged, minced, and incubated in a 0.075% collagenase in D-PBS solution for 30-90 minutes. The solution was neutralized with MSC media, containing Advanced MEM (Gibco/Life Technologies, Grand Island, N.Y.), GlutaMAX (Gibco/Life Technologies), PLTMax (Mill Creek Life Sciences, Rochester, Minn.), and heparin. The cells were cultured and expanded on BD Falcon cell culture flasks in MSC media. Samples were directly collected (Control MSCs), and the equivalent was added to GORE® BIO-A® Fistula plugs (matrix) and incubated four additional days prior to collection.

Next generation RNA-seq was performed on the TruSeq platform (Illumina, San Diego, Calif.) using high quality RNA that was purified using oligo dT magnetic beads as described elsewhere (Dudakovic et al., J. Biol. Chem., 288:28783-28791 (2013)). The resulting fraction enriched for poly A mRNAs was subjected to first and second strand cDNA synthesis using random primers, followed by ligation to paired-end DNA adaptors with unique barcodes (Sets A and B) (Illumina) for flow cell multiplexing. Paired-end reads obtained using Illumina HiSeq 2000 were subjected to a standard bioinformatic pipeline for base-calling (Illumina's RTA version 1.17.21.3), and a raw RNA-sequencing data analysis system (MAPRSeq v.1.2.1) that includes read alignment (TopHat 2.0.6), gene counting (HTSeq software), and expression analysis were performed using edgeR 2.6.2. Reads per kilobasepair per million mapped reads (RPKM) were compared for MSCs from six different patients grown on plastic or GORE® BIO-A® Fistula plugs. Differences in gene expression were determined using a paired Student's t-test, as well as rank-ordering for P-values, RPKMs and fold changes in control MSCs versus MSCs grown on GORE® BIO-A® Fistula plugs. Tables and graphs were prepared using Excel (Microsoft Office), and hierarchical clustering was performed with GENE-E (Broad Institute, Boston, Mass.). Gene ontology analyses were performed using DAVID6.7, FunRich, Reactome and GeneMania, as well as focused PubMed searches for genes that were incompletely annotated.

### Results

### Growth Kinetics, Phenotype, and Characterization of Cells Used for Therapy

The protocol proved highly feasible with every patient biopsy capable of generating a viable clinical product. One patient required re-collection of adipose tissue due to contamination. Cells grew rapidly with average doublings of 1.5 per day (after second plating). The protocol administered live, recently bound cells to a matrix. Release testing was done at the time of cryopreservation. Post thaw viability was routinely above 95%. Cells were counted in the supernatant during cell binding to properly understand the dose of cells on the matrix. For all samples, less than 5% of the cells remained in the supernatant on completion of the incubation confirming their ability to recover and grow well following storage. Patient MSCs universally demonstrated the classic MSC phenotype with CD44, CD73, CD105 and Class I positivity, and CD14, CD45 and Class II negativity.

### Efficacy and Safety

Twenty patients were screened for study enrollment, of which 12 were treated. Patients enrolled had persistent refractory disease (median of 5 years of perianal disease, and an average of 5.5 prior exams under anesthesia for treatment). All patients were on biologic therapy at the time of study enrollment, and all remained on the same biologic therapy at six months following MSC-matrix placement.

There were three serious adverse events, none of which were related to Crohn's disease or placement of the MSC-matrix, and none of which led to study withdrawal. There were two non-serious adverse events related to seromas at the site of fat collection. There were an additional 15 non-serious events, of which 9 were non-serious adverse events related to underlying Crohn's disease, and 6 were non-serious adverse events not related to underlying Crohn's disease or the study interventions.

Nine of 12 patients had complete clinical healing by 3 months, and ten of 12 patients (83%) had complete clinical healing at six months. Of the two patients without clinical healing, one developed an abscess at three months requiring drainage and seton placement, and the other had persistent drainage from a new ramification off the original fistula, resulting in an anolabial fistula. No patients experienced incontinence or the need to wear pads for leakage by six months. A total of four patients (33%) received a less than 30-day course of antibiotics due to new symptoms or findings on an interval MRI study. No patients had a change in the medical management of their Crohn's disease.

MRI was used to clearly define the characteristics of the treated fistula tracts at baseline and six months. Radiographic criteria for treatment response was demonstrated in 10 of 12 patients (83%). Overall, there was a significant decrease in the length of T2-weighted hyperintensity within the fistula tract (median decrease 22%, range −5 to 100%, p=0.01), and a non-significant decrease in diameter (median decrease 57%, range −36 to 100%, p=0.27), with negative values representing an increase in fistula size in the two treatment failures. Van Assche perianal severity scores also decreased significantly (median 13 to median 9, p=0.0008), without worsening in any of the patients: one treatment failure had an unchanged score of 21 owing to supralevator extension and small abscess at baseline (with no change in extension and another small abscess after treatment), and the other failure had an unchanged score of 12, with an abscess resolving but the hyperintense fistula tract increasing in size.

Scatter plots of changes in length and diameter of T2-weighted hyperintensity within the fistula tract and Van Assche scores at baseline and at 6-month follow-up MR are shown in FIGS. 12A-B. In the 10 responding patients, Van Assche scores decreased in 9, with the single patient with no change in Van Assche score demonstrating response with substantial decrease in length and diameter of T2-hyperintensity a branching transsphincteric fistula. Additionally, mean absolute changes for length and diameter of fistula tract decreased by a mean of 23.5 and 5.0 mm, respectively, in responding patients, and increased by a mean of 0.2 and 10 mm in the two treatment failures, respectively. One treatment failure demonstrated rectal inflammation and a 12 mm abscess on the pre-procedural MRI, and at the six month MRI demonstrated a continuing abscess with increase in size of branching ramifications. The second treatment failure demonstrated a patent internal opening on follow-up MRI and increase diameter of the fistula, potentially indicating displacement of the MSC fistula plug.

### Matrix Bound MSCs Exhibit an Altered Gene Expression Signature

To understand the biological properties of the effect of MSCs bound to a Gore® Bio-A® Fistula Plug, RNA-seq analysis was performed to determine the expression levels of protein coding mRNAs for all annotated genes (n=23,338) in MSCs grown on regular polystyrene tissue-culture plastic (‘control’; n=6)) versus the Gore® Bio-A® Fistula Plug (‘matrix’; n=6). Dot-plot analysis revealed that the overall RNA expression patterns in both control and matrix samples were comparable in both experimental conditions. However, hierarchical clustering of the entire RNA-seq dataset for all twelve samples (filtered for RPKM expression value>0.3) showed that control and matrix MSCs form two distinct biological clades with characteristic gene expression patterns specific to control and matrix MSCs (FIGS. 13A-B).

To define these specific gene signatures, the lists of all genes that were robustly expressed in either biological condition (RPKM>0.3) and statistically different between control and matrix MSCs (p<0.05) were intersected. There were 898 and 165 genes uniquely detected in control and matrix MSCs, respectively. Of the 11,548 genes commonly expressed in both conditions, more than half (n=6,131) exhibited statistical differences in expression (FIGS. 13A-B). Thus, culturing MSCs on a biomaterial matrix (i.e., Gore Bio-A® Fistula Plug) resulted in prominent modulations in gene expression. GSEA was subsequently performed to focus upon upregulated gene networks associated with matrix adherence and physiologically relevant to optimized function.

Examination of genes in both control and matrix MSCs revealed that the most highly expressed mRNAs encode cytoplasmic and/or cytoskeletal proteins (FIG. 14A). More germane to the function of MSCs in generating a cellularized implant for fistula repair, ECM proteins were well-represented. This set included the non-collagenous proteins fibronectin (FN) and osteonectin (SPARC), as well as collagen types I, III, VI and V (COL1A1, COL1A2, COL3A1, COL6A1, COL6A2, COL5A1, COL5A2)(Figures 14A and 14B). Even though collagens I, III, VI and V were most highly expressed in matrix MSCs (RPKM>100), mRNAs for COL15A1, COL10A1, COL8A2 and COL9A2 exhibited the largest fold-change when MSCs were grown on the fistula matrix (>10 fold). The latter non-fibrillary collagens were only expressed at moderate levels (between 5 and 70 RPKM) (FIG. 14B). Importantly, analysis of the relative expression of all collagen genes (n=43) relative to all other annotated genes (n=23,338) showed that even though collagens represent only 0.18% of all genes, they accounted for approximately 6% of all mRNAs expressed in MSCs (FIG. 14B).

To assess whether MSCs have the potential for ECM remodeling, the expression of matrix metalloproteinase genes (MMPs) was examined in MSCs grown on the fistula matrix (i.e., Gore® Bio-A® Fistula Plug). Heat map analysis and numerical sorting of expression values revealed that matrix MSCs exhibited elevated expression of several highly expressed MMPs, such as MMP1, −2, −3, −13 and −14. Expression of these and other ECM remodeling enzymes may facilitate integration of a collagen-embedded and MSC-enhanced implant into patients for fistula repair.

Matrix MSCs have a quiescent and protein anabolic cellular phenotype. To define the biological activity and phenotypic molecular signature of matrix MSCs, a gene ontology analysis was performed (FIG. 15). The most abundant protein coding transcripts expressed in control MSCs were genes generally related to the cell cycle, mitosis, proliferation and/or pro-oncogenic pathways (n=23 within the top 25). In contrast, the most abundant genes selectively enriched in matrix MSCs were those encoding glycoproteins and/or integral membrane proteins (n=21 within the top 25). Broader analysis of all genes selectively expressed and statistically different in control and matrix MSCs revealed that genes linked to the cell cycle were depleted while genes supporting protein translation were enriched in matrix MSCs.

To understand whether growth of MSCs on matrix alters their secretory properties, expression data for a list of 285 genes encoding known cytokines, growth factors, morphogens, ligand inhibitors and other protein ligands were selected. The list of genes was generated based on gene ontology terms and focused literature surveys. Of this gene set, there were 52 genes (e.g., CCL3, IL2, BMP15, FGF4, WNT3A) that were not detected at all (RPKM=0) and 113 genes (e.g., CCL1, IL3, BMP3, FGF3, WNT4) that were expressed below an arbitrary threshold (RPKM<0.3) in both samples. Of the remaining 120 genes for secreted proteins, only twelve proteins were detected that were selectively upregulated by at least two-fold (RPKM>0.3; P<0.05 based on paired T-test).

Additionally, there were fifteen genes encoding secreted factors that were down regulated by two-fold when MSCs were grown on matrix (RPKM>0.3; P<0.05 based on paired T-test). The most prominent protein was the TGFβ target gene CTGF, which encodes connective tissue growth factor.

These results demonstrate that the methods and materials provided herein can produce a biologic that is distinct from the cells added during incubation, and that this new biologic has powerful therapeutic capable of repairing fistulas.

### Example 3—Stem Cells on Matrix Plugs

In another experiment, MSCs were grown on different types of matrices, and the expression of various polypeptides was assessed and compared to the expression level exhibited by MSCs grown in media alone. The results were provided in FIGS. 16A-D and FIGS. 17A-D.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a fistula in a mammal, wherein said method comprises implanting a scaffold into said fistula, wherein said scaffold comprises fibers and mesenchymal stem cells located between said fibers, wherein said fibers comprise polymers of polyglycolic acid and trimethylene carbonate.

2. The method of claim 1, wherein said mammal is a human.

3. The method of claim 1, wherein said fistula is an anal fistula.

4-6. (canceled)

7. The method of claim 1, wherein said polyglycolic acid is about 60 to about 70 percent of said fibers.

8. The method of claim 1, wherein said polyglycolic acid is about 67 percent of said fibers.

9. The method of claim 1, wherein said trimethylene carbonate is about 30 to about 40 percent of said fibers.

10. The method of claim 1, wherein said trimethylene carbonate is about 33 percent of said fibers.

11. The method of claim 1, wherein said scaffold comprises platelet derivative material.

12. The method of claim 1, wherein said fibers are randomly arranged fibers.

13. A method for making an implant for treating a fistula, wherein said method comprises contacting a scaffold comprises fibers with mesenchymal stem cells within a polypropylene container, wherein said fibers comprise polymers of polyglycolic acid and trimethylene carbonate.

14-17. (canceled)

18. The method of claim 13, wherein said polyglycolic acid is about 60 to about 70 percent of said fibers.

19. The method of claim 13, wherein said polyglycolic acid is about 67 percent of said fibers.

20. The method of claim 13, wherein said trimethylene carbonate is about 30 to about 40 percent of said fibers.

21. The method of claim 13, wherein said trimethylene carbonate is about 33 percent of said fibers.

22. The method of claim 13, wherein said method comprises contacting said scaffold with platelet derivative material within said container.

23. (canceled)

24. A scaffold comprising fibers and mesenchymal stem cells located between said fibers, wherein said fibers comprise polymers of polyglycolic acid and trimethylene carbonate, and wherein said mesenchymal stem cells express more fibroblast growth factor 2 (FGF-2) polypeptide, eotaxin polypeptide, FMS-like tyrosine kinase 3 ligand (FLT3L) polypeptide, growth-regulated protein (GRO) polypeptide, and interleukin 10 (IL-10) polypeptide than comparable mesenchymal stem cells cultured in the absence of said fibers, and wherein said mesenchymal stem cells express less fractalkine polypeptide than said comparable mesenchymal stem cells.

25. (canceled)

26. The scaffold of claim 24, wherein said polyglycolic acid is about 60 to about 70 percent of said fibers.

27. The scaffold of claim 24, wherein said polyglycolic acid is about 67 percent of said fibers.

28. The scaffold of claim 24, wherein said trimethylene carbonate is about 30 to about 40 percent of said fibers.

29. The scaffold of claim 24, wherein said trimethylene carbonate is about 33 percent of said fibers.

30-34. (canceled)
